(19) United States
(12) Patent Application Publication
(10) Pub. No.: US 2023/0346041 A1
(43) Pub. Date: Nov. 2, 2023

(54) CONSUMABLE IDENTIFICATION AND POWER CONTROL

(71) Applicant: RAI STRATEGIC HOLDINGS, INC., WINSTON-SALEM, NC (US)

(72) Inventors: Jason Michael Short, Winston-Salem, NC (US); Zhihuang Xiao, Eastleigh (GB)

(21) Appl. No.: 17/661,443

(22) Filed: Apr. 29, 2022

(51) Int. Cl. A24F 40/53; A24F 40/42; A24F 40/57

(52) U.S. Cl. CPC A24F 40/53; A24F 40/42; A24F 40/57

(57) ABSTRACT

An aerosol provision system includes a consumable with an aerosol generator and aerosol-generating material, and an aerosol provision device to control power provided to the aerosol generator to energize the aerosol-generating material to generate aerosol. The consumable is one of a plurality of types of consumable having target values of an electrical characteristic that are associated with respective power modes of different levels of power for different amounts of aerosol generated from the aerosol-generating material. The aerosol provision device measures the electrical characteristic of the consumable to obtain a value, and identifies one of the target values that most closely matches the value. The one of the target values is associated with one of the power modes of a respective one of the different levels of power. And the aerosol provision device controls the power provided to the aerosol generator to the respective one of the different levels of power.

### TECHNOLOGICAL FIELD

The present disclosure relates to aerosol provision systems such as smoking articles designed to deliver at least one substance to a user.

### BACKGROUND

Many aerosol provision systems and in particular non-combustible aerosol provision systems have been proposed through the years as improvements upon, or alternatives to, smoking products that require combusting tobacco for use. These systems are generally designed to deliver at least one substance to a user, such as to satisfy a particular “consumer moment.” To this end, the substance may include constituents that impart a physiological effect on the user, a sensorial effect on the user, or both. The substance may be generally present in an aerosol-generating material that may contain one or more constituents of a range of constituents, such as active substances, flavors, aerosol-former materials and other functional materials like fillers.

Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs). Many of these products take the form of a system including a device and a consumable, and it is the consumable that includes the material from which the substance to be delivered originates. Typically, the device is reusable, and the consumable is single-use (although some consumables are refillable). Therefore, in many cases, the consumable is sold separately from the device, and often in a multipack. Moreover, subsystems and some individual components of devices or consumables may be sourced from specialist manufacturers.

### BRIEF SUMMARY

Example implementations of the present disclosure are directed to aerosol provision systems such as smoking articles designed to deliver at least one substance to a user. The present disclosure includes, without limitation, the following example implementations.

Some example implementations provide an aerosol provision system comprising: a consumable with an aerosol generator and aerosol-generating material, the consumable one of a plurality of types of consumable having target values of an electrical characteristic that are associated with respective power modes of different levels of power for different amounts of aerosol generated from the aerosol-generating material; and an aerosol provision device structured to engage the consumable, and configured to control power provided to the aerosol generator to energize the aerosol-generating material of the consumable to generate aerosol for delivery to a user, including the aerosol provision device configured to at least: measure the electrical characteristic of the consumable to obtain a value of the electrical characteristic; identify one of the target values that most closely matches the value of the electrical characteristic of the consumable, the one of the target values associated with one of the power modes of a respective one of the different levels of power for a respective one of the different amounts of aerosol; and control the power provided to the aerosol generator to the respective one of the different levels of power for the respective one of the different amounts of aerosol.

Some example implementations provide an aerosol provision device comprising: a coupler structured to engage a consumable with an aerosol generator and aerosol-generating material, the consumable one of a plurality of types of consumable having target values of an electrical characteristic that are associated with respective power modes of different levels of power for different amounts of aerosol generated from the aerosol-generating material; and circuitry that includes processing circuitry configured to control power provided to the aerosol generator to energize the aerosol-generating material of the consumable to generate aerosol for delivery to a user, including the processing circuitry configured to at least: measure the electrical characteristic of the consumable to obtain a value of the electrical characteristic; identify one of the target values that most closely matches the value of the electrical characteristic of the consumable, the one of the target values associated with one of the power modes of a respective one of the different levels of power for a respective one of the different amounts of aerosol; and control the power provided to the aerosol generator to the respective one of the different levels of power for the respective one of the different amounts of aerosol.

Some example implementations provide a method of operating an aerosol provision device that includes a coupler structured to engage a consumable with an aerosol generator and aerosol-generating material, the consumable one of a plurality of types of consumable having target values of an electrical characteristic that are associated with respective power modes of different levels of power for different amounts of aerosol generated from the aerosol-generating material, the method comprising: controlling power provided to the aerosol generator to energize the aerosol-generating material of the consumable to generate aerosol for delivery to a user, including at least: measuring the electrical characteristic of the consumable to obtain a value of the electrical characteristic; identifying one of the target values that most closely matches the value of the electrical characteristic of the consumable, the one of the target values associated with one of the power modes of a respective one of the different levels of power for a respective one of the different amounts of aerosol; and controlling the power provided to the aerosol generator to the respective one of the different levels of power for the respective one of the different amounts of aerosol.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURES

Having thus described aspects of the disclosure in the foregoing general terms, reference will now be made to the accompanying figures, which are not necessarily drawn to scale, and wherein:

FIG. 1 is a block diagram of an aerosol provision system according to some example implementations of the present disclosure;

FIGS. 2 and 3 illustrate an aerosol provision system in the form of a vapor product, according to some example implementations;

FIGS. 4A and 4B illustrate first and second type of consumable for use in an aerosol provision system, according to some example implementations;

FIG. 5 illustrates circuitry of an aerosol provision device, according to some example implementations;

FIGS. 6, 7, 8 and 9 illustrate lumped-element models of a consumable, according to various example implementations;

FIGS. 10A, 10B, 10C, 10D, 10E, 10F, 10G and 10H are flowcharts illustrating various steps in a method of operating an aerosol provision device, according to various example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably.

Example implementations of the present disclosure are generally directed to delivery systems designed to deliver at least one substance to a user, such as to satisfy a particular “consumer moment.” The substance may include constituents that impart a physiological effect on the user, a sensorial effect on the user, or both.

Delivery systems may take many forms. Examples of suitable delivery systems include aerosol provision systems such as powered aerosol provision systems designed to release one or more substances or compounds from an aerosol-generating material without combusting the aerosol-generating material. These aerosol provision systems may at times be referred to as non-combustible aerosol provision systems, aerosol delivery devices or the like, and the aerosol-generating material may be, for example, in the form of a solid, semi-solid, liquid or gel and may or may not contain nicotine.

Examples of suitable aerosol provision systems include vapor products, heat-not-burn products, hybrid products and the like. Vapor products are commonly known as “electronic cigarettes,” “e-cigarettes” or electronic nicotine delivery systems (ENDS), although the aerosol-generating material need not include nicotine. Many vapor products are designed to heat a liquid material to generate an aerosol. Other vapor products are designed to break up an aerosol-generating material into an aerosol without heating, or with only secondary heating. Heat-not-burn products include tobacco heating products (THPs) and carbon-tipped tobacco heating products (CTHPs), and many are designed to heat a solid material to generate an aerosol without combusting the material.

Hybrid products use a combination of aerosol-generating materials, one or a plurality of which may be heated. Each of the aerosol-generating materials may be, for example, in the form of a solid, semi-solid, liquid, or gel. Some hybrid products are similar to vapor products except that the aerosol generated from a liquid or gel aerosol-generating material passes through a second material (such as tobacco) to pick up additional constituents before reaching the user. In some example implementations, the hybrid system includes a liquid or gel aerosol-generating material, and a solid aerosol-generating material. The solid aerosol-generating material may include, for example, tobacco or a non-tobacco product.

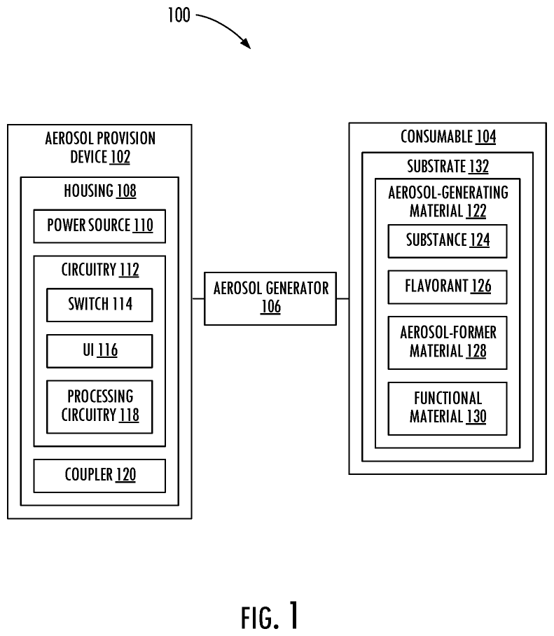

FIG. 1 is a block diagram of an aerosol provision system 100 according to some example implementations. In various examples, the aerosol provision system may be a vapor product, heat-not-burn product or hybrid product. The aerosol provision system includes one or more of each of a number of components including, for example, an aerosol provision device 102, and a consumable 104 (sometimes referred to as an article) for use with the aerosol provision device. The aerosol provision system also includes an aerosol generator 106. In various implementations, the aerosol generator may be part of the aerosol provision device or the consumable. In other implementations, the aerosol generator may be separate from the aerosol provision device and the consumable, and removably engaged with the aerosol provision device and/or the consumable.

In various examples, the aerosol provision system 100 and its components including the aerosol provision device 102 and the consumable 104 may be reusable or single-use. In some examples, the aerosol provision system including both the aerosol provision device and the consumable may be single use. In some examples, the aerosol provision device may be reusable, and the consumable may be reusable (e.g., refillable) or single use (e.g., replaceable). In yet further examples, the consumable may be both refillable and also replaceable. In examples in which the aerosol generator 106 is part of the aerosol provision device or the consumable, the aerosol generator may be reusable or single-use in the same manner as the aerosol provision device or the consumable.

In some example implementations, the aerosol provision device 102 may include a housing 108 with a power source 110 and circuitry 112. The power source is configured to provide a source of power to the aerosol provision device and thereby the aerosol provision system 100. The power source may be or include, for example, an electric power source such as a non-rechargeable battery or a rechargeable battery, solid-state battery (SSB), lithium-ion battery, supercapacitor, or the like.

The circuitry 112 may be configured to enable one or more functionalities (at times referred to as services) of the aerosol provision device 102 and thereby the aerosol provision system 100. The circuitry includes electronic components, and in some examples one or more of the electronic components may be formed as a circuit board such as a printed circuit board (PCB).

In some examples, the circuitry 112 includes at least one switch 114 that may be directly or indirectly manipulated by a user to activate the aerosol provision device 102 and thereby the aerosol provision system 100. The switch may be or include a pushbutton, touch-sensitive surface or the like that may be operated manually by a user. Additionally or alternatively, the switch may be or include a sensor configured to sense one or more process variables that indicate use of the aerosol provision device or aerosol provision system. One example is a flow sensor, pressure sensor, pressure switch or the like that is configured to detect airflow or a change in pressure caused by airflow when a user draws on the consumable 104.

The switch 114 may provide user interface functionality. In some examples, the circuitry 112 may include a user interface (UI) 116 that is separate from or that is or includes the switch. The UI may include one or more input devices and/or output devices to enable interaction between the user and the aerosol provision device 102. As described above with respect to the switch, examples of suitable input devices include pushbuttons, touch-sensitive surfaces and the like. The one or more output devices generally include devices configured to provide information in a human-perceptible form that may be visual, audible or tactile/haptic. Examples of suitable output devices include light sources such as light-emitting diodes (LEDs), quantum dot-based LEDs and the like. Other examples of suitable output devices include display devices (e.g., electronic visual displays), touchscreens (integrated touch-sensitive surface and display device), loudspeakers, vibration motors and the like.

In some examples, the circuitry 112 includes processing circuitry 118 configured to perform data processing, application execution, or other processing, control or management services according to one or more example implementations. The processing circuitry may include a processor embodied in a variety of forms such as at least one processor core, microprocessor, coprocessor, controller, microcontroller or various other computing or processing devices including one or more integrated circuits such as, for example, an ASIC (application specific integrated circuit), an FPGA (field programmable gate array), some combination thereof, or the like. In some examples, the processing circuitry may include memory coupled to or integrated with the processor, and which may store data, computer program instructions executable by the processor, some combination thereof, or the like.

As also shown, in some examples, the housing 108 and thereby the aerosol provision device 102 may also include a coupler 120 structured to engage and hold the consumable 104, and thereby couple the aerosol provision device with the consumable. The coupler may be or include a connector, fastener or the like that is configured to connect with a corresponding coupler of the consumable, such as by a press fit (or interference fit) connection, threaded connection, magnetic connection or the like. The coupler may be or include a receptacle such as a reservoir, tank, container, cavity, receiving chamber or the like that is structured to receive and contain the consumable or at least a portion of the consumable.

The consumable 104 is an article including aerosol-generating material 122 (also referred to as an aerosol precursor composition), part or all of which is intended to be consumed during use by a user. The aerosol provision system 100 may include one or more consumables, and each consumable may include one or more aerosol-generating materials. In some examples in which the aerosol provision system is a hybrid product, the aerosol provision system may include a liquid or gel aerosol-generating material to generate an aerosol, which may then pass through a second, solid aerosol-generating material to pick up additional constituents before reaching the user. These aerosol-generating materials may be within a single consumable or respective consumables that may be separately removable.

The aerosol-generating material 122 is capable of generating aerosol, for example when heated, irradiated or energized in any other way. The aerosol-generating material may be, for example, in the form of a solid, semi-solid, liquid or gel. The aerosol-generating material may include an “amorphous solid,” which may be alternatively referred to as a “monolithic solid” (i.e., non-fibrous). In some examples, the amorphous solid may be a dried gel. The amorphous solid is a solid material that may retain some fluid, such as liquid, within it. In some examples, the aerosol-generating material may include from about 50 wt %, 60 wt % or 70 wt % of amorphous solid, to about 90 wt %, 95 wt % or 100 wt % of amorphous solid.

The aerosol-generating material 122 may include one or more of each of a number of constituents such as an active substance 124, flavorant 126, aerosol-former material 128 or other functional material 130.

The active substance 124 may be a physiologically active material, which is a material intended to achieve or enhance a physiological response such as improved alertness, improved focus, increased energy, increased stamina, increased calm or improved sleep. The active substance may for example be selected from nutraceuticals, nootropics, psychoactives. The active substance may be naturally occurring or synthetically obtained. The active substance may include, for example, nicotine, caffeine, GABA (γ-aminobutyric acid), L-theanine, taurine, theine, vitamins such as B6 or B12 (cobalamin) or C, melatonin, cannabinoids, terpenes, or constituents, derivatives, or combinations thereof. The active substance may include one or more constituents, derivatives or extracts of tobacco, cannabis or another botanical.

In some examples in which the active substance 124 includes derivatives or extracts, the active substance may be or include one or more cannabinoids or terpenes.

As noted herein, the active substance 124 may include or be derived from one or more botanicals or constituents, derivatives or extracts thereof. As used herein, the term “botanical” includes any material derived from plants including, but not limited to, extracts, leaves, bark, fibers, stems, roots, seeds, flowers, fruits, pollen, husk, shells or the like. Alternatively, the material may include an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens.

In yet other examples, the active substance 124 may be or include one or more of 5-hydroxytryptophan (5-HTP)/oxitriptan/Griffonia simplicifolia, acetylcholine, arachidonic acid (AA, omega-6), ashwagandha (Withania somnifera), Bacopa monniera, beta alanine, beta-hydroxy-beta-methylbutyrate (HMB), Centella asiatica, chai-hu, cinnamon, citicoline, cotinine, creatine, curcumin, docosahexaenoic acid (DHA, omega-3), dopamine, Dorstenia arifolia, Dorstenia Odorata, essential oils, GABA, Galphimia glauca, glutamic acid, hops, kaempferia parviflora (Thai ginseng), kava, L-carnitine, L-arginine, lavender oil, L-choline, liquorice, L-lysine, L-theanine, L-tryptophan, lutein, magnesium, magnesium L-threonate, myo-inositol, nardostachys chinensis, nitrate, oil-based extract of Viola odorata, oxygen, phenylalanine, phosphatidylserine, quercetin, resveratrol, Rhizoma gastrodiae, Rhodiola, Rhodiola rosea, rose essential oil, S-adenosylmethionine (SAMe), sceletium tortuosum, schisandra, selenium, serotonin, skullcap, spearmint extract, spikenard, theobromine, tumaric, Turnera aphrodisiaca, tyrosine, vitamin A, vitamin B3, or yerba mate.

In some example implementations, the aerosol-generating material 122 includes a flavorant 126. As used herein, the terms “flavorant” and “flavor” refer to materials which, where local regulations permit, may be used to create a desired taste, aroma or other somatosensorial sensation in a product for adult consumers. Flavorants may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, redberry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. Flavorants may be imitation, synthetic or natural ingredients or blends thereof. Flavorants may be in any suitable form, for example, liquid such as an oil, solid such as a powder, or gas.

In some example implementations, the flavorant 126 may include a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

The aerosol-former material 128 may include one or more constituents capable of forming an aerosol. In some example implementations, the aerosol-former material may include one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials 130 may include one or more of pH regulators, colouring agents, preservatives, binders, fillers, stabilizers, and/or antioxidants. Suitable binders include, for example, pectin, guar gum, fruit pectin, citrus pectin, tobacco pectin, hydroxyethyl guar gum, hydroxypropyl guar gum, hydroxyethyl locust bean gum, hydroxypropyl locust bean gum, alginate, starch, modified starch, derivatized starch, methyl cellulose, ethyl cellulose, ethylhydroxymethyl cellulose, carboxymethyl cellulose, tamarind gum, dextran, pullalon, konjac flour or xanthan gum.

In some example implementations, the aerosol-generating material 122 may be present on or in a support to form a substrate 132. The support may be or include, for example, paper, card, paperboard, cardboard, reconstituted material (e.g., a material formed from reconstituted plant material, such as reconstituted tobacco, reconstituted hemp, etc.), a plastics material, a ceramic material, a composite material, glass, a metal, or a metal alloy. In some examples, the support includes a susceptor, which may be embedded within the aerosol-generating material, or on one or either side of the aerosol-generating material.

Although not separately shown, in some example implementations, the consumable 104 may further include a receptacle structured to engage and hold the aerosol-generating material 122, or substrate 132 with the aerosol-generating material. The receptacle may be or include a reservoir, tank, container, cavity, receiving chamber or the like that is structured to receive and contain the aerosol-generating material or the substrate. The consumable may include an aerosol-generating material transfer component (also referred to as a liquid transport element) configured to transport aerosol-generating material to the aerosol generator 106. The aerosol-generating material transfer component may be adapted to wick or otherwise transport aerosol-generating material via capillary action. In some examples, the aerosol-generating material transfer component may include a microfluidic chip, a micro pump or other suitable component to transport aerosol-generating material.

The aerosol generator 106 (also referred to as an atomizer, aerosolizer or aerosol production component) is configured to energize the aerosol-generating material 122 to generate an aerosol, or otherwise cause generation of an aerosol from the aerosol-generating material. More particularly, in some examples, the aerosol generator may be powered by the power source 110 under control of the circuitry 112 to energize the aerosol-generating material to generate an aerosol.

In some example implementations, the aerosol generator 106 is an electric heater configured to perform electric heating in which electrical energy from the power source is converted to heat energy, which the aerosol-generating material is subject to so as to release one or more volatiles from the aerosol-generating material to form an aerosol. Examples of suitable forms of electric heating include resistance (Joule) heating, induction heating, dielectric and microwave heating, radiant heating, arc heating and the like. More particular examples of suitable electric heaters include resistive heating elements such as wire coils, flat plates, prongs, micro heaters or the like.

In some example implementations, the aerosol generator 106 is configured to cause an aerosol to be generated from the aerosol-generating material without heating, or with only secondary heating. For example, the aerosol generator may be configured to subject the aerosol-generating material to one or more of increased pressure, vibration, or electrostatic energy. More particular examples of these aerosol generators include jet nebulizers, ultrasonic wave nebulizers, vibrating mesh technology (VMT) nebulizers, surface acoustic wave (SAW) nebulizers, and the like.

A jet nebulizer is configured to use compressed gas (e.g., air, oxygen) to break up aerosol-generating material 122 into an aerosol, and an ultrasonic wave nebulizer is configured to use ultrasonic waves to break up aerosol-generating material into an aerosol. A VMT nebulizer includes a mesh, and a piezo material (e.g., piezoelectric material, piezomagnetic material) that may be driven to vibrate and cause the mesh to break up aerosol-generating material into an aerosol. A SAW nebulizer is configured to use surface acoustic waves or Rayleigh waves to break up aerosol-generating material into an aerosol.

In some examples, the aerosol generator 106 may include a susceptor, or the susceptor may be part of the substrate 132. The susceptor is a material that is heatable by penetration with a varying magnetic field generated by a magnetic field generator that may be separate from or part of the aerosol generator. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor in some examples may be both electrically-conductive and magnetic, so that the susceptor of these examples is heatable by both heating mechanisms.

Although not separately shown, either or both the aerosol provision device 102 or the consumable 104 may include an aerosol-modifying agent. The aerosol-modifying agent is a substance configured to modify the aerosol generated from the aerosol-generating material 122, such as by changing the taste, flavor, acidity or another characteristic of the aerosol. In various examples, the aerosol-modifying agent may be an additive or a sorbent. The aerosol-modifying agent may include, for example, one or more of a flavorant, colorant, water or carbon adsorbent. The aerosol-modifying agent may be a solid, semi-solid, liquid or gel. The aerosol-modifying agent may be in powder, thread or granule form. The aerosol-modifying agent may be free from filtration material. In some examples, the aerosol-modifying agent may be provided in an aerosol-modifying agent release component that is operable to selectively release the aerosol-modifying agent.

The aerosol provision system 100 and its components including the aerosol provision device 102, consumable 104, and aerosol generator 106 may be manufactured with any of a number of different form factors, and with additional or alternative components relative to those described above.

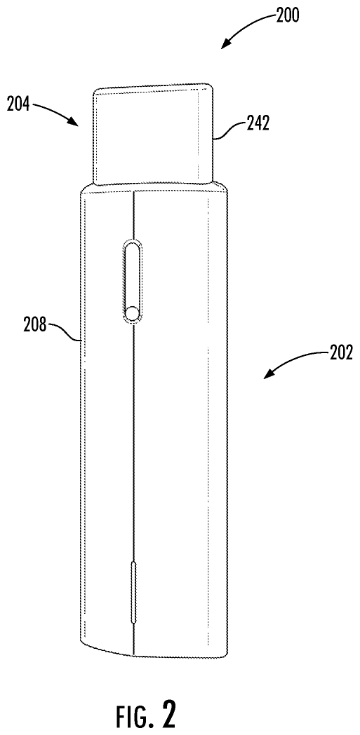

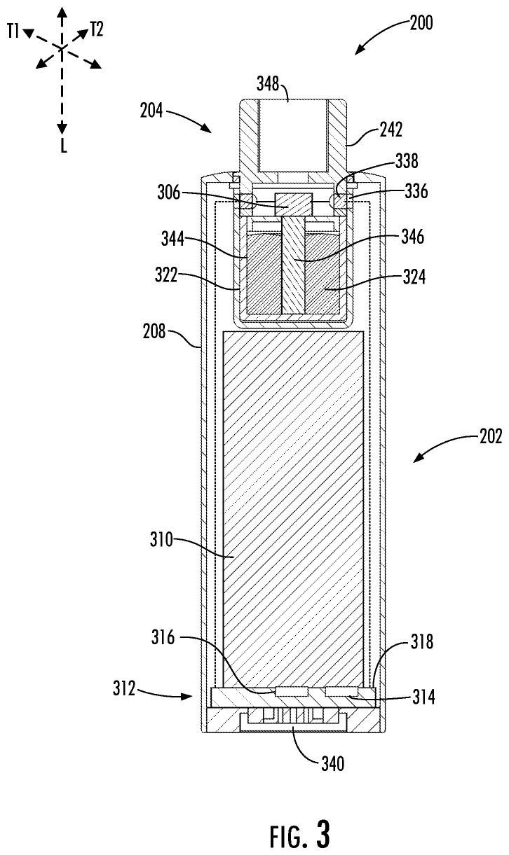

FIGS. 2 and 3 illustrate an aerosol provision system 200 in the form of a vapor product, and that in some example implementations may correspond to the aerosol provision system 100. As shown, the aerosol provision system 200 may include an aerosol provision device 202 (also referred to as a control body or power unit) and a consumable 204 (also referred to as a cartridge or tank), which may correspond to respectively the aerosol provision device 102 and the consumable 104. The aerosol provision system and in particular the consumable may also include an aerosol generator corresponding to the aerosol generator 106, and in the form of an electric heater 306 such as a heating element like a metal wire coil configured to convert electrical energy to heat energy through resistance (Joule) heating. The aerosol provision device and the consumable can be permanently or detachably aligned in a functioning relationship. FIGS. 2 and 3 illustrate respectively a perspective view and a partially cut-away side view of the aerosol provision system in a coupled configuration.

As seen in FIG. 2 and the cut-away view illustrated in FIG. 3, the aerosol provision device 202 and consumable 204 each include a number of respective components. The components illustrated in FIG. 3 are representative of the components that may be present in an aerosol provision device and consumable and are not intended to limit the scope of components that are encompassed by the present disclosure.

The aerosol provision device 202 may include a housing 208 (sometimes referred to as an aerosol provision device shell) that may include a power source 310. The housing may also include circuitry 312 with a switch in the form of a sensor 314, a user interface including a light source 316 that may be illuminated with use of the aerosol provision system 200, and processing circuitry 318 (also referred to as a control component). The housing may also include a receptacle in the form of a consumable receiving chamber 322 structured to engage and hold the consumable 204. And the consumable may include an aerosol-generating material 324 that may correspond to aerosol-generating material 122, and that may include one or more of each of a number of constituents such as an active substance, flavorant, aerosol-former material or other functional material.

As also seen in FIG. 3, the aerosol provision device 202 may also include electrical connectors 336 positioned in the consumable receiving chamber 322 configured to electrically couple the circuitry and thereby the aerosol provision device with the consumable 204, and in particular electrical contacts 338 on the consumable. In this regard, the electrical connectors and electrical contacts may form a connection interface of the aerosol provision device and consumable. As also shown, the aerosol provision device may include an external electrical connector 340 to connect the aerosol provision device with one or more external devices. Examples of suitable external electrical connectors include USB connectors, proprietary connectors such as Apple's Lightning connector, and the like.

In various examples, the consumable 204 includes a tank portion and a mouthpiece portion. The tank portion and the mouthpiece portion may be integrated or permanently fixed together, or the tank portion may itself define the mouthpiece portion (or vice versa). In other examples, the tank portion and the mouthpiece portion may be separate and removably engaged with one another.

The consumable 204, tank portion and/or mouthpiece portion may be separately defined in relation to a longitudinal axis (L), a first transverse axis (T1) that is perpendicular to the longitudinal axis, and a second transverse axis (T2) that is perpendicular to the longitudinal axis and is perpendicular to the first transverse axis. The consumable can be formed of a housing 242 (sometimes referred to as the consumable shell) enclosing a reservoir 344 (in the tank portion) configured to retain the aerosol-generating material 324. In some examples, the consumable may include an aerosol generator, such as electric heater 306 in the illustrated example. In some examples, the electrical connectors 336 on the aerosol provision device 202 and electrical contacts 338 on the consumable may electrically connect the electric heater with the power source 310 and/or circuitry 312 of the aerosol provision device.

As shown, in some examples, the reservoir 344 may be in fluid communication with an aerosol-generating material transfer component 346 adapted to wick or otherwise transport aerosol-generating material 324 stored in the reservoir housing to the electric heater 306. At least a portion of the aerosol-generating material transfer component may be positioned proximate (e.g., directly adjacent, adjacent, in close proximity to, or in relatively close proximity to) the electric heater. The aerosol-generating material transfer component may extend between the electric heater and the aerosol-generating material stored in the reservoir, and at least a portion of the electric heater may be located above a proximal end the reservoir. For the purposes of the present disclosure, it should be understood that the term “above” in this particular context should be interpreted as meaning toward a proximal end of the reservoir and/or the consumable 204 in direction substantially along the longitudinal axis (L). Other arrangements of the aerosol-generating material transfer component are also contemplated within the scope of the disclosure. For example, in some example implementations, the aerosol-generating material transfer component may be positioned proximate a distal end of the reservoir and/or arranged transverse to the longitudinal axis (L).

The electric heater 306 and aerosol-generating material transfer component 346 may be configured as separate elements that are fluidly connected, the electric heater and aerosol-generating material transfer component or may be configured as a combined element. For example, in some implementations an electric heater may be integrated into an aerosol-generating material transfer component. Moreover, the electric heater and the aerosol-generating material transfer component may be formed of any construction as otherwise described herein. In some examples, a valve may be positioned between the reservoir 344 and electric heater, and configured to control an amount of aerosol-generating material 324 passed or delivered from the reservoir to the electric heater.

An opening 348 may be present in the housing 242 (e.g., at the mouth end of the mouthpiece portion) to allow for egress of formed aerosol from the consumable 204.

As indicated above, the circuitry 312 of the aerosol provision device 202 may include a number of electronic components, and in some examples may be formed of a circuit board such as a PCB that supports and electrically connects the electronic components. The sensor 314 (switch) may be one of these electronic components positioned on the circuit board. In some examples, the sensor may comprise its own circuit board or other base element to which it can be attached. In some examples, a flexible circuit board may be utilized. A flexible circuit board may be configured into a variety of shapes. In some examples, a flexible circuit board may be combined with, layered onto, or form part or all of a heater substrate.

In some examples, the reservoir 344 may be a container for storing the aerosol-generating material 324. In some examples, the reservoir may be or include a fibrous reservoir with a substrate with the aerosol-generating material present on or in a support. For example, the reservoir can comprise one or more layers of nonwoven fibers substantially formed into the shape of a tube encircling the interior of the housing 242, in this example. The aerosol-generating material may be retained in the reservoir. Liquid components, for example, may be sorptively retained by the reservoir. The reservoir may be in fluid connection with the aerosol-generating material transfer component 346. The aerosol-generating material transfer component may transport the aerosol-generating material stored in the reservoir via capillary action—or via a micro pump—to the electric heater 306. As such, the electric heater is in a heating arrangement with the aerosol-generating material transfer component.

In use, when a user draws on the aerosol provision system 200, airflow is detected by the sensor 314, and the electric heater 306 is activated to energize the aerosol-generating material 324 to generate an aerosol. Drawing upon the mouth end of the aerosol provision system causes ambient air to enter and pass through the aerosol provision system. In the consumable 204, the drawn air combines with the aerosol that is whisked, aspirated or otherwise drawn away from the electric heater and out the opening 348 in the mouth end of the aerosol provision system.

According to some example implementations of the present disclosure, a consumable 104, 204 may be one of a plurality of types of consumable. The aerosol-generating material 122, 324 may be the same or different across the plurality of types of consumable. In some examples, the plurality of types of consumable may be externally identical but differ in volume of the aerosol-generating material that the plurality of types of consumable are structured to receive.

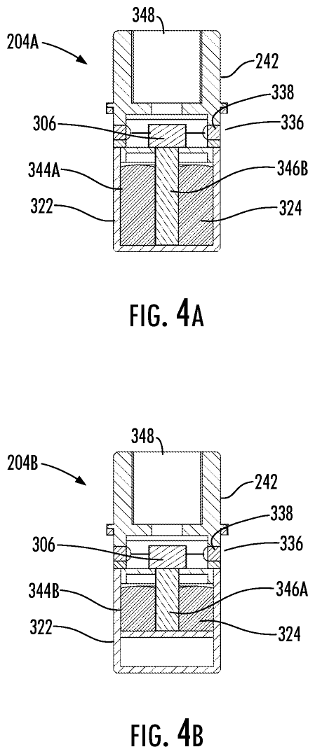

FIG. 4A illustrates a first type of consumable 204A that corresponds to the consumable 204 shown in FIGS. 2 and 3, according to some example implementations. Similarly, FIG. 4B illustrates a second type of consumable 204B, according to some example implementations. As shown, the first and second types of consumable are externally identical but differ in volume of aerosol-generating material 324 that their respective reservoirs 344A, 344B are structured to receive. In this regard, the first type of consumable may be structured to receive a first volume of the aerosol-generating material, and the second type of consumable may be structured to receive a second volume of the aerosol-generating material; and the first volume of the aerosol-generating material may be greater than the second volume. As also shown, the first and second types of consumable may include respective aerosol-generating material transfer components 346A, 346B that are sized according to their respective reservoirs.

According to example implementations, the plurality of types of consumable 104, 204 may also differ in the value of an electrical characteristic such as electrical resistance, current, voltage or other electrical characteristic that describes the emission, behavior or effect of electrons caused by the consumable. The plurality of types of consumable may therefore have target values of an electrical characteristic, and the target values are associated with respective power modes of different levels of power for different amounts of aerosol generated from the aerosol-generating material. In examples in which the aerosol generator 106 is a resistive heating element, the different levels of power may be for different temperatures to which the resistive heating element is driven to generate the different amounts of aerosol, which may be measured in terms of aerosol collected mass (ACM) or total particulate matter (TPM).

In various examples, the target value of an electrical characteristic may be a value or a range of values. One example of a suitable range of values is reflected by a target value +/− an acceptable tolerance from the target value. The target values of the electrical characteristic of the consumables may be defined so that the target values are sufficiently different from one another so that the aerosol provision device may identify the consumable or otherwise distinguish between the different types of consumables.

The processing circuitry 118 of the aerosol provision device 102 may be configured to identify a consumable 104 (e.g., consumable 204), and control power provided to the aerosol generator 106 based on the type of the consumable. The aerosol generator may thereby be powered to energize the aerosol-generating material 122 of the consumable 104 to generate aerosol for delivery to a user, which may be responsive to user input that indicates a draw on the consumable.

In some more particular examples, the processing circuitry 118 may be configured to measure the electrical characteristic of a consumable 104 to obtain a value of the electrical characteristic, and identify one of the target values that most closely matches the value of the electrical characteristic of the consumable. The target values may be set with a buffer between them that reduces a likelihood that an unintended one of the target values is identified from the electrical resistance measured by the processing circuitry. Additionally or alternatively, the processing circuitry may make multiple measurements of the value of the electrical characteristic, identify one of the target values for respective ones of the multiple measurements, and only proceed when the one of the target values is the same for the multiple measurements.

The one of the target values that is identified may be associated with one of the power modes of a respective one of the different levels of power for a respective one of the different amounts of aerosol. The processing circuitry 118 may then control the power provided to the aerosol generator 106 to the respective one of the different levels of power for the respective one of the different amounts of aerosol. In some examples, the processing circuitry may be configured to set the aerosol provision device to the one of the power modes before power is provided to the aerosol generator.

In some examples, the control of power to the aerosol generator 106 may include the processing circuitry 118 configured to measure a voltage across and a current through the aerosol generator. The processing circuitry may calculate the power provided to the aerosol generator based on the voltage and the current, and adjust the power provided to the aerosol generator at each instance in which the power as calculated deviates from the respective one of the levels by more than a threshold power.

In more particular examples, calculation of the power provided to the aerosol generator 106 may include the processing circuitry 118 configured to determine a moving window of measurements of instantaneous actual power provided to the aerosol generator, each measurement of the window of measurements determined based on the voltage and the current. The processing circuitry may then calculate the power provided to the aerosol generator as a simple moving average power based on the moving window of measurements instantaneous actual power.

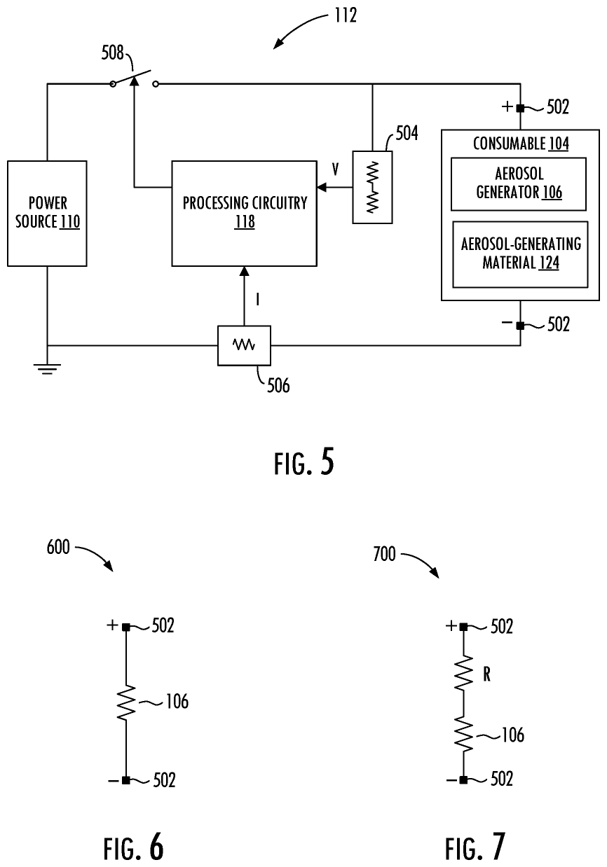

FIG. 5 illustrates circuitry 112 of the aerosol provision device 102, according to some example implementations. In this regard, in some examples, the coupler 120 includes electrical connectors 502 (e.g., electrical connectors 336) configured to electrically couple the circuitry and thereby the aerosol provision device with the consumable 104. In some of these examples, the processing circuitry may be configured to measure the electrical characteristic of the consumable at the electrical connectors.

In some examples, the processing circuitry 118 may be configured to measure at least one of a voltage across or a current through the consumable 104 at the electrical connectors 502, and determine the value of the electrical characteristic of the consumable based on the at least one of the voltage or the current. In particular, the processing circuitry may be configured to measure the voltage across the consumable from a positive one of the electrical connectors and referenced to ground, such as through a voltage divider 504. Similarly, the processing circuitry may be configured to measure the current through the consumable using a low-side current-sensing circuit 506 coupled to a negative one of the electrical connectors.

As also shown in FIG. 5, the circuitry 112 may include a switch 508 such as a high-side load switch by which the processing circuitry may be configured to switchably connect and disconnect the power source and the aerosol generator, and thereby control the power provided to the aerosol generator includes.

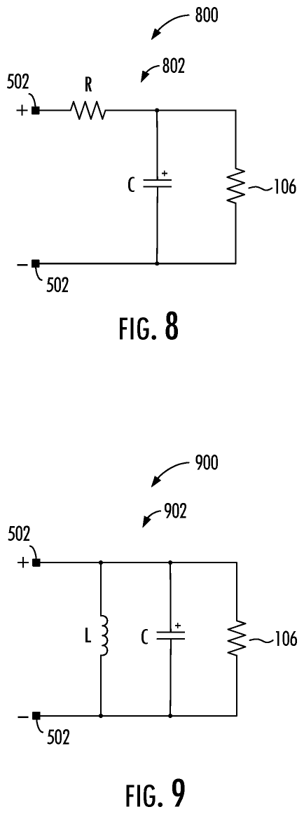

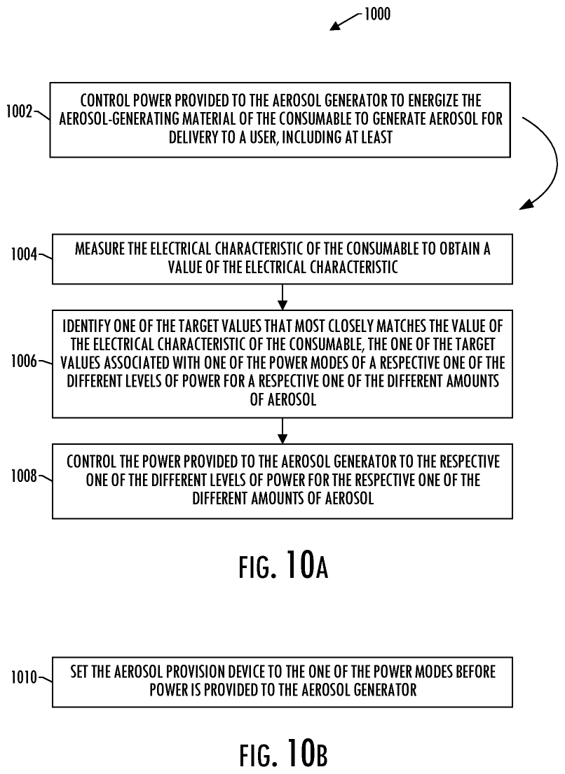

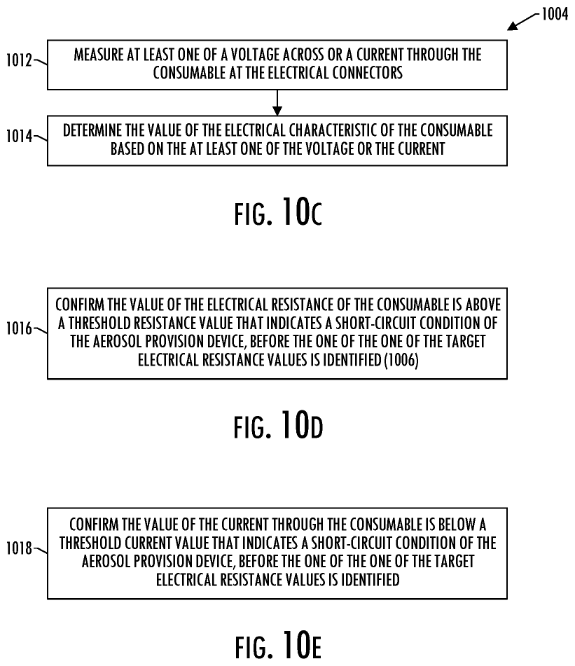

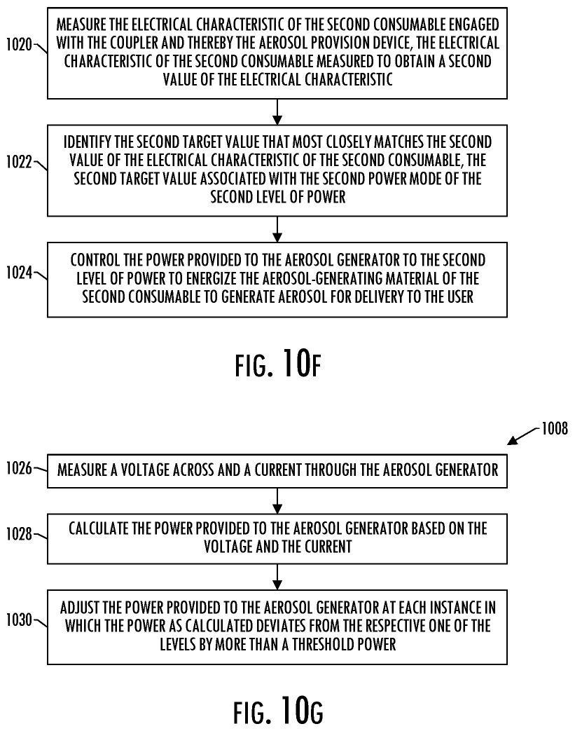

FIGS. 6, 7, 8 and 9 illustrate lumped-element models 600, 700, 800 and 900 of a consumable 104, according to various example implementations in which the aerosol generator 106 is a resistive heating element. As shown in lumped-element model 600, the electrical resistance of the consumable may be the electrical resistance of the resistive heating element. In lumped-element model 700, the consumable further includes a series resistor R, and the electrical resistance of the consumable is a sum of the electrical resistance of the resistive heating element and the series resistor. In lumped-element model 800, the consumable further includes a resistor-capacitor (RC) circuit 802 that includes the resistive heating element; and in lumped-element model 900, the consumable further includes a resistor-inductor-capacitor (RLC) circuit 902 that includes the resistive heating element.

In various examples in which the electrical characteristic is electrical resistance, the value of the electrical characteristic is a value of the electrical resistance of the consumable 104. This may be the electrical resistance of the resistive heating element in lumped-element model 600 of the consumable. Similarly, the value of the electrical resistance of the consumable may be the sum of the electrical resistance of the resistive heating element and the series resistor R in lumped-element model 700.

The processing circuitry 118 may be configured to measure the voltage across and the current through the consumable 104, and determine the value of the electrical resistance of the consumable based on the voltage and the current. This may include the processing circuitry configured to control power from the power source 110 to apply a voltage across the consumable for a short duration (e.g., 20 milliseconds), measure the voltage across and a current through the consumable, and determine the electrical resistance from the voltage and the current. Second, after a valid consumable 104 is read, the processing circuitry 118 may wait a duration (e.g., 5 milliseconds), repeat the process to determine the electrical resistance, and only proceed if the same electrical resistance is determined on both attempts.

In examples in which the electrical characteristic is electrical resistance, the processing circuitry 118 may be configured to confirm the value of the electrical resistance of the consumable 104 is above a threshold resistance value that indicates a short-circuit condition of the aerosol provision device 102, before the one of the one of the target electrical resistance values is identified. The processing circuitry may therefore confirm the value of the electrical resistance across the electrical connectors 502 as a short-circuit protection measure before power is controlled to the electrical connectors.

In some examples in which the electrical characteristic is current, the value of the electrical characteristic may be a value of the current through the consumable 104. This may be the current through the resistive heating element in lumped-element model 600 of the consumable, or the current through the resistive heating element and the series resistor R in lumped-element model 700. Similar to electrical resistance, the current may also be used for short-circuit protection. In this regard, the processing circuitry 118 may be configured to confirm the value of the current through the consumable is below a threshold current value that indicates a short-circuit condition of the aerosol provision device.

In other examples in which the electrical characteristic is current, the value of the electrical characteristic may be a waveform of the current through the consumable 104. The waveform of the current through the consumable may be a waveform of the current through the RC circuit 802 in lumped-element model 800 of the consumable. Or the waveform of the current may be a waveform of the current through the RLC circuit 902 in lumped-element model 900 of the consumable.

To further illustrate some example implementations of the present disclosure, consider the consumable 122 such as consumable 204A that is of a first of the plurality of types of consumable having a first target value of the electrical characteristic associated with a first power mode of a first level of power. Similarly, consider a second consumable such as consumable 204B that is of a second of the plurality of types of consumable having a second target value of the electrical characteristic associated with a second power mode of a second level of power.

In some of these examples, the processing circuitry 118 may be configured to measure the electrical characteristic, identify the first target value, and control the power provided to the aerosol generator 106, for the consumable 104. Similarly, the processing circuitry may be configured to measure the electrical characteristic of the second consumable engaged with the coupler 120 and thereby the aerosol provision device. The electrical characteristic of the second consumable is measured to obtain a second value of the electrical characteristic, and the processing circuitry may identify the second target value that most closely matches the second value of the electrical characteristic of the second consumable. The second target value is associated with the second power mode of the second level of power, and the processing circuitry may control the power provided to the aerosol generator to the second level of power to energize the aerosol-generating material of the second consumable to generate aerosol for delivery to the user.

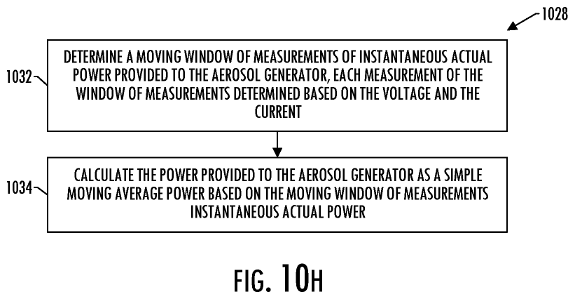

FIGS. 10A-10H are flowcharts illustrating various steps in a method 1000 of operating an aerosol provision device, according to various example implementations of the present disclosure. As described above, the aerosol provision device includes a coupler structured to engage a consumable with an aerosol generator and aerosol-generating material. The consumable is one of a plurality of types of consumable having target values of an electrical characteristic that are associated with respective power modes of different levels of power for different amounts of aerosol generated from the aerosol-generating material. The method includes controlling power provided to the aerosol generator to energize the aerosol-generating material of the consumable to generate aerosol for delivery to a user, as shown at block 1002 of FIG. 10A.

In particular, controlling the power to the aerosol generator includes measuring the electrical characteristic of the consumable to obtain a value of the electrical characteristic, as shown at block 1004. The method includes identifying one of the target values that most closely matches the value of the electrical characteristic of the consumable, the one of the target values associated with one of the power modes of a respective one of the different levels of power for a respective one of the different amounts of aerosol, as shown at block 1006. And the method includes controlling the power provided to the aerosol generator to the respective one of the different levels of power for the respective one of the different amounts of aerosol, as shown at block 1008.

In some examples, the method 1000 further includes setting the aerosol provision device to the one of the power modes before power is provided to the aerosol generator, as shown at block 1010 of FIG. 10B.

In some examples, the power provided to the aerosol generator is controlled at block 1002 responsive to user input that indicates a draw on the consumable.

In some examples, the coupler includes electrical connectors configured to electrically couple the aerosol provision device with the consumable. In some of these examples, the electrical characteristic of the consumable is measured at block 1004 at the electrical connectors.

In some examples, measuring the electrical characteristic of the consumable at block 1004 includes measuring at least one of a voltage across or a current through the consumable at the electrical connectors, as shown at block 1012 of FIG. 10C. And the method includes determining the value of the electrical characteristic of the consumable based on the at least one of the voltage or the current, as shown at block 1014.

In some examples, the voltage across the consumable is measured at block 1012 from a positive one of the electrical connectors and referenced to ground.

In some examples, the current through the consumable is measured at block 1012 using a low-side current-sensing circuit coupled to a negative one of the electrical connectors.

In some examples, the electrical characteristic is electrical resistance, and the value of the electrical characteristic is a value of the electrical resistance of the consumable. In some of these examples, the voltage across and the current through the consumable are measured at block 1012, and the value of the electrical resistance of the consumable is determined at block 1014 based on the voltage and the current.

In some examples, the electrical characteristic is electrical resistance, the target values are target electrical resistance values, the value of the electrical characteristic is a value of the electrical resistance of the consumable, and the one of the target values is one of the target electrical resistance values. In some of these examples, the method 1000 further includes confirming the value of the electrical resistance of the consumable is above a threshold resistance value that indicates a short-circuit condition of the aerosol provision device, before the one of the one of the target electrical resistance values is identified at block 1006, as shown at block 1016 of FIG. 10D.

In some examples, the aerosol generator is a resistive heating element, and the electrical resistance of the consumable is the electrical resistance of the resistive heating element in a lumped-element model of the consumable.

In some examples, the aerosol generator is a resistive heating element, the consumable further includes a series resistor, and the electrical resistance of the consumable is a sum of the electrical resistance of the resistive heating element and the series resistor in a lumped-element model of the consumable.

In some examples, the electrical characteristic is current, the target values are target current values, the value of the electrical characteristic is a value of the current through the consumable, and the one of the target values is one of the target current values. In some of these examples, the method 1000 further includes confirming the value of the current through the consumable is below a threshold current value that indicates a short-circuit condition of the aerosol provision device, before the one of the one of the target electrical resistance values is identified, as shown at block 1018 of FIG. 10E.

In some examples, the consumable is of a first of the plurality of types of consumable having a first target value of the electrical characteristic associated with a first power mode of a first level of power, a second consumable of a second of the plurality of types of consumable having a second target value of the electrical characteristic associated with a second power mode of a second level of power.

In some examples, the first of the plurality of types of consumable is structured to receive a first volume of the aerosol-generating material, and the second of the plurality of types of consumable is structured to receive a second volume of the aerosol-generating material. In some of these examples, the first volume of the aerosol-generating material is greater than the second volume, and the first value of the power setting is greater than the second value.

In some examples, the method 1000 further includes measuring the electrical characteristic of the second consumable engaged with the coupler and thereby the aerosol provision device, the electrical characteristic of the second consumable measured to obtain a second value of the electrical characteristic, as shown at block 1020 of FIG. 10F. The method includes identifying the second target value that most closely matches the second value of the electrical characteristic of the second consumable, the second target value associated with the second power mode of the second level of power, as shown at block 1022. And the method includes controlling the power provided to the aerosol generator to the second level of power to energize the aerosol-generating material of the second consumable to generate aerosol for delivery to the user, as shown at block 1024.

In some examples, the aerosol provision device further comprises a power source configured to provide a source of power to the aerosol provision device. In some of these examples, controlling the power provided to the aerosol generator at block 1008 inductees switchably connecting and disconnecting the power source and the aerosol generator.

In some examples, the controlling the power provided to the aerosol generator at block 1008 includes measuring a voltage across and a current through the aerosol generator, as shown at block 1026 of FIG. 10G. The method includes calculating the power provided to the aerosol generator based on the voltage and the current, as shown at block 1028. And the method includes adjusting the power provided to the aerosol generator at each instance in which the power as calculated deviates from the respective one of the levels by more than a threshold power, as shown at block 1030.

In some examples, calculating the power provided to the aerosol generator at block 1028 includes determining a moving window of measurements of instantaneous actual power provided to the aerosol generator, each measurement of the window of measurements determined based on the voltage and the current, as shown at block 1032 of FIG. 1011, And the method includes calculating the power provided to the aerosol generator as a simple moving average power based on the moving window of measurements instantaneous actual power, as shown at block 1034.

As explained above and reiterated below, the present disclosure includes, without limitation, the following example implementations.
Clause 1. An aerosol provision system comprising: a consumable with an aerosol generator and aerosol-generating material, the consumable one of a plurality of types of consumable having target values of an electrical characteristic that are associated with respective power modes of different levels of power for different amounts of aerosol generated from the aerosol-generating material; and an aerosol provision device structured to engage the consumable, and configured to control power provided to the aerosol generator to energize the aerosol-generating material of the consumable to generate aerosol for delivery to a user, including the aerosol provision device configured to at least: measure the electrical characteristic of the consumable to obtain a value of the electrical characteristic; identify one of the target values that most closely matches the value of the electrical characteristic of the consumable, the one of the target values associated with one of the power modes of a respective one of the different levels of power for a respective one of the different amounts of aerosol; and control the power provided to the aerosol generator to the respective one of the different levels of power for the respective one of the different amounts of aerosol.
Clause 2. The aerosol provision system of clause 1, wherein the aerosol-generating material is the same across the plurality of types of consumable.
Clause 3. The aerosol provision system of clause 1 or clause 2, wherein the plurality of types of consumable are externally identical but differ in the electrical characteristic, and in volume of the aerosol-generating material that the plurality of types of consumable are structured to receive.
Clause 4, The aerosol provision system of any of clauses 1 to 3, wherein the different levels of power are for the different amounts of aerosol that are measured in terms of aerosol collected mass (ACM) or total particulate matter (TPM).
Clause 5. The aerosol provision system of any of clauses 1 to 4, wherein the aerosol generator is a resistive heating element, and the different levels of power are for different temperatures to which the resistive heating element is driven to generate the different amounts of aerosol.
Clause 6. The aerosol provision system of any of clauses 1 to 5, wherein the aerosol provision device is further configured to set the aerosol provision device to the one of the power modes before power is provided to the aerosol generator.
Clause 7. The aerosol provision system of any of clauses 1 to 6, wherein the aerosol provision device is configured to control the power provided to the aerosol generator responsive to user input that indicates a draw on the consumable.
Clause 8. The aerosol provision system of any of clauses 1 to 7, wherein the aerosol provision device includes electrical connectors configured to electrically couple the aerosol provision device with the consumable, and wherein the aerosol provision device is configured to measure the electrical characteristic of the consumable at the electrical connectors.
Clause 9, The aerosol provision system of clause 8, wherein the aerosol provision device configured to measure the electrical characteristic of the consumable includes the aerosol provision device configured to at least: measure at least one of a voltage across or a current through the consumable at the electrical connectors; and determine the value of the electrical characteristic of the consumable based on the at least one of the voltage or the current.
Clause 10. The aerosol provision system of clause 9, wherein the aerosol provision device is configured to measure the voltage across the consumable from a positive one of the electrical connectors and referenced to ground.
Clause 11. The aerosol provision system of clause 9 or clause 10, wherein the aerosol provision device is configured to measure the current through the consumable using a low-side current-sensing circuit coupled to a negative one of the electrical connectors.
Clause 12. The aerosol provision system of any of clauses 9 to 11, wherein the electrical characteristic is electrical resistance, and the value of the electrical characteristic is a value of the electrical resistance of the consumable, and wherein the aerosol provision device is configured to measure the voltage across and the current through the consumable, and determine the value of the electrical resistance of the consumable based on the voltage and the current.
Clause 13. The aerosol provision system of any of clauses 1 to 12, wherein the electrical characteristic is electrical resistance, the target values are target electrical resistance values, the value of the electrical characteristic is a value of the electrical resistance of the consumable, and the one of the target values is one of the target electrical resistance values.
Clause 14. The aerosol provision system of clause 13, wherein the aerosol provision device is further configured to confirm the value of the electrical resistance of the consumable is above a threshold resistance value that indicates a short-circuit condition of the aerosol provision device, before the one of the one of the target electrical resistance values is identified.
Clause 15. The aerosol provision system of clause 13 or clause 14, wherein the aerosol generator is a resistive heating element, and the electrical resistance of the consumable is the electrical resistance of the resistive heating element in a lumped-element model of the consumable.
Clause 16. The aerosol provision system of any of clauses 13 to 15, wherein the aerosol generator is a resistive heating element, the consumable further includes a series resistor, and the electrical resistance of the consumable is a sum of the electrical resistance of the resistive heating element and the series resistor in a lumped-element model of the consumable.
Clause 17. The aerosol provision system of any of clauses 1 to 16, wherein the electrical characteristic is current, the target values are target current values, the value of the electrical characteristic is a value of the current through the consumable, and the one of the target values is one of the target current values.
Clause 18. The aerosol provision system of clause 17, wherein the aerosol provision device is further configured to confirm the value of the current through the consumable is below a threshold current value that indicates a short-circuit condition of the aerosol provision device, before the one of the one of the target electrical resistance values is identified.
Clause 19. The aerosol provision system of clause 17 or clause 18, wherein the aerosol generator is a resistive heating element, and the current through the consumable is the current through the resistive heating element in a lumped-element model of the consumable.
Clause 20. The aerosol provision system of any of clauses 1 to 19, wherein the electrical characteristic is current, the target values are target current waveforms, the value of the electrical characteristic is a waveform of the current through the consumable, and the one of the target values is one of the target current waveforms.
Clause 21. The aerosol provision system of clause 20, wherein the aerosol generator is a resistive heating element, the consumable further includes a resistor-capacitor (RC) circuit that includes the resistive heating element, and the waveform of the current through the consumable is a waveform of the current through the RC circuit in a lumped-element model of the consumable.
Clause 22. The aerosol provision system of clause 20 or clause 21, wherein the aerosol generator is a resistive heating element, the consumable further includes a resistor-inductor-capacitor (RLC) circuit that includes the resistive heating element, and the waveform of the current through the consumable is a waveform of the current through the RLC circuit in a lumped-element model of the consumable.
Clause 23. The aerosol provision system of any of clauses 1 to 22, wherein the consumable is of a first of the plurality of types of consumable having a first target value of the electrical characteristic associated with a first power mode of a first level of power, a second consumable of a second of the plurality of types of consumable having a second target value of the electrical characteristic associated with a second power mode of a second level of power.
Clause 24. The aerosol provision system of clause 23, wherein the first of the plurality of types of consumable is structured to receive a first volume of the aerosol-generating material, and the second of the plurality of types of consumable is structured to receive a second volume of the aerosol-generating material.
Clause 25. The aerosol provision system of clause 24, wherein the first volume of the aerosol-generating material is greater than the second volume, and the first value of the power setting is greater than the second value.
Clause 26. The aerosol provision system of any of clauses 23 to 25, wherein the aerosol provision device is further configured to at least: measure the electrical characteristic of the second consumable engaged with the aerosol provision device, the electrical characteristic of the second consumable measured to obtain a second value of the electrical characteristic; identify the second target value that most closely matches the second value of the electrical characteristic of the second consumable, the second target value associated with the second power mode of the second level of power; and control the power provided to the aerosol generator to the second level of power to energize the aerosol-generating material of the second consumable to generate aerosol for delivery to the user.
Clause 27. The aerosol provision system of any of clauses 1 to 26, wherein the aerosol provision device further comprises a power source configured to provide a source of power to the aerosol provision device, and wherein the aerosol provision device configured to control the power provided to the aerosol generator includes the aerosol provision device configured to switchably connect and disconnect the power source and the aerosol generator.
Clause 28. The aerosol provision system of any of clauses 1 to 27, wherein the aerosol provision device configured to control the power provided to the aerosol generator includes the aerosol provision device configured to at least: measure a voltage across and a current through the aerosol generator; calculate the power provided to the aerosol generator based on the voltage and the current; and adjust the power provided to the aerosol generator at each instance in which the power as calculated deviates from the respective one of the levels by more than a threshold power,
Clause 29. The aerosol provision system of clause 28, wherein the aerosol provision device configured to calculate the power provided to the aerosol generator includes the aerosol provision device configured to at least: determine a moving window of measurements of instantaneous actual power provided to the aerosol generator, each measurement of the window of measurements determined based on the voltage and the current; and calculate the power provided to the aerosol generator as a simple moving average power based on the moving window of measurements instantaneous actual power.
Clause 30. An aerosol provision device comprising: a coupler structured to engage a consumable with an aerosol generator and aerosol-generating, material, the consumable one of a plurality of types of consumable having target values of an electrical characteristic that are associated with respective power modes of different levels of power for different amounts of aerosol generated from the aerosol-generating material; and circuitry that includes processing circuitry configured to control power provided to the aerosol generator to energize the aerosol-generating material of the consumable to generate aerosol for delivery to a user, including the processing circuitry configured to at least: measure the electrical characteristic of the consumable to obtain a value of the electrical characteristic; identify one of the target values that most closely matches the value of the electrical characteristic of the consumable, the one of the target values associated with one of the power modes of a respective one of the different levels of power for a respective one of the different amounts of aerosol; and control the power provided to the aerosol generator to the respective one of the different levels of power for the respective one of the different amounts of aerosol.
Clause 31. The aerosol provision device of clause 30, wherein the aerosol-generating material is the same across the plurality of types of consumable.
Clause 32. The aerosol provision device of clause 30 or clause 31, wherein the plurality of types of consumable are externally identical but differ in the electrical characteristic, and in volume of the aerosol-generating material that the plurality of types of consumable are structured to receive.
Clause 31 The aerosol provision device of any of clauses 30 to 32, wherein the different levels of power are for the different amounts of aerosol that are measured in terms of aerosol collected mass (ACM) or total particulate matter (TPM).
Clause 34. The aerosol provision device of any of clauses 30 to 33, wherein the aerosol generator is a resistive heating element, and the different levels of power are for different temperatures to which the resistive heating element is driven to generate the different amounts of aerosol.
Clause 35. The aerosol provision device of any of clauses 30 to 34, wherein the processing circuitry is further configured to set the aerosol provision device to the one of the power modes before power is provided to the aerosol generator.
Clause 36. The aerosol provision device of any of clauses 30 to 35, wherein the processing circuitry is configured to control the power provided to the aerosol generator responsive to user input that indicates a draw on the consumable.
Clause 37. The aerosol provision device of any of clauses 30 to 36, wherein the coupler includes electrical connectors configured to electrically couple the circuitry and thereby the aerosol provision device with the consumable, and wherein the processing circuitry is configured to measure the electrical characteristic of the consumable at the electrical connectors.
Clause 38. The aerosol provision device of clause 37, wherein the processing circuitry configured to measure the electrical characteristic of the consumable includes the processing circuitry configured to at least: measure at least one of a voltage across or a current through the consumable at the electrical connectors; and determine the value of the electrical characteristic of the consumable based on the at least one of the voltage or the current.
Clause 39. The aerosol provision device of clause 38, wherein the processing circuitry is configured to measure the voltage across the consumable from a positive one of the electrical connectors and referenced to ground.
Clause 40. The aerosol provision device of clause 38 or clause 39, wherein the processing circuitry is configured to measure the current through the consumable using a low-side current-sensing circuit coupled to a negative one of the electrical connectors.
Clause 41. The aerosol provision device of any of clauses 38 to 40, wherein the electrical characteristic is electrical resistance, and the value of the electrical characteristic is a value of the electrical resistance of the consumable, and wherein the processing circuitry is configured to measure the voltage across and the current through the consumable, and determine the value of the electrical resistance of the consumable based on the voltage and the current.
Clause 42. The aerosol provision device of any of clauses 30 to 41, wherein the electrical characteristic is electrical resistance, the target values are target electrical resistance values, the value of the electrical characteristic is a value of the electrical resistance of the consumable, and the one of the target values is one of the target electrical resistance values.
Clause 43. The aerosol provision device of clause 42, wherein the processing circuitry is further configured to confirm the value of the electrical resistance of the consumable is above a threshold resistance value that indicates a short-circuit condition of the aerosol provision device, before the one of the one of the target electrical resistance values is identified.
Clause 44. The aerosol provision device of clause 42 or clause 43, wherein the aerosol generator is a resistive heating element, and the electrical resistance of the consumable is the electrical resistance of the resistive heating element in a lumped-element model of the consumable.
Clause 45. The aerosol provision device of any of clauses 42 to 44, wherein the aerosol generator is a resistive heating element, the consumable further includes a series resistor, and the electrical resistance of the consumable is a sum of the electrical resistance of the resistive heating element and the series resistor in a lumped-element model of the consumable.
Clause 46. The aerosol provision device of any of clauses 30 to 45, wherein the electrical characteristic is current, the target values are target current values, the value of the electrical characteristic is a value of the current through the consumable, and the one of the target values is one of the target current values.
Clause 47. The aerosol provision device of clause 46, wherein the processing circuitry is further configured to confirm the value of the current through the consumable is below a threshold current value that indicates a short-circuit condition of the aerosol provision device, before the one of the one of the target electrical resistance values is identified.
Clause 48. The aerosol provision device of clause 46 or clause 47, wherein the aerosol generator is a resistive heating element, and the current through the consumable is the current through the resistive heating element in a lumped-element model of the consumable.
Clause 49. The aerosol provision device of any of clauses 30 to 48, wherein the electrical characteristic is current, the target values are target current waveforms, the value of the electrical characteristic is a waveform of the current through the consumable, and the one of the target values is one of the target current waveforms.
Clause 50. The aerosol provision device of clause 49, wherein the aerosol generator is a resistive heating element, the consumable further includes a resistor-capacitor (RC) circuit that includes the resistive heating element, and the waveform of the current through the consumable is a waveform of the current through the RC circuit in a lumped-element model of the consumable.
Clause 51. The aerosol provision device of clause 49 or clause 50, wherein the aerosol generator is a resistive heating element, the consumable further includes a resistor-inductor-capacitor (RLC) circuit that includes the resistive heating element, and the waveform of the current through the consumable is a waveform of the current through the RLC circuit in a lumped-element model of the consumable.
Clause 52. The aerosol provision device of any of clauses 30 to 51, wherein the consumable is of a first of the plurality of types of consumable having a first target value of the electrical characteristic associated with a first power mode of a first level of power, a second consumable of a second of the plurality of types of consumable having a second target value of the electrical characteristic associated with a second power mode of a second level of power.
Clause 53. The aerosol provision device of clause 52, wherein the first of the plurality of types of consumable is structured to receive a first volume of the aerosol-generating material, and the second of the plurality of types of consumable is structured to receive a second volume of the aerosol-generating material.
Clause 54. The aerosol provision device of clause 53, wherein the first volume of the aerosol-generating material is greater than the second volume, and the first value of the power setting is greater than the second value.
Clause 55. The aerosol provision device of any of clauses 52 to 54, wherein the processing circuitry is further configured to at least: measure the electrical characteristic of the second consumable engaged with the coupler and thereby the aerosol provision device, the electrical characteristic of the second consumable measured to obtain a second value of the electrical characteristic; identify the second target value that most closely matches the second value of the electrical characteristic of the second consumable, the second target value associated with the second power mode of the second level of power; and control the power provided to the aerosol generator to the second level of power to energize the aerosol-generating material of the second consumable to generate aerosol for delivery to the user.
Clause 56. The aerosol provision device of any of clauses 30 to 55, wherein the aerosol provision device further comprises a power source configured to provide a source of power to the aerosol provision device, and wherein the processing circuitry configured to control the power provided to the aerosol generator includes the processing circuitry configured to switchably connect and disconnect the power source and the aerosol generator.
Clause 57. The aerosol provision device of any of clauses 30 to 56, wherein the processing circuitry configured to control the power provided to the aerosol generator includes the processing circuitry configured to at least: measure a voltage across and a current through the aerosol generator; calculate the power provided to the aerosol generator based on the voltage and the current; and adjust the power provided to the aerosol generator at each instance in which the power as calculated deviates from the respective one of the levels by more than a threshold power,
Clause 58. The aerosol provision device of clause 57, wherein the processing circuitry configured to calculate the power provided to the aerosol generator includes the processing circuitry configured to at least: determine a moving window of measurements of instantaneous actual power provided to the aerosol generator, each measurement of the window of measurements determined based on the voltage and the current; and calculate the power provided to the aerosol generator as a simple moving average power based on the moving window of measurements instantaneous actual power.
Clause 59. A method of operating an aerosol provision device that includes a coupler structured to engage a consumable with an aerosol generator and aerosol-generating material, the consumable one of a plurality of types of consumable having target values of an electrical characteristic that are associated with respective power modes of different levels of power for different amounts of aerosol generated from the aerosol-generating material, the method comprising: controlling power provided to the aerosol generator to energize the aerosol-generating material of the consumable to generate aerosol for delivery to a user, including at least: measuring the electrical characteristic of the consumable to obtain a value of the electrical characteristic; identifying one of the target values that most closely matches the value of the electrical characteristic of the consumable, the one of the target values associated with one of the power modes of a respective one of the different levels of power for a respective one of the different amounts of aerosol; and controlling the power provided to the aerosol generator to the respective one of the different levels of power for the respective one of the different amounts of aerosol.
Clause 60. The method of clause 59, wherein the aerosol-generating material is the same across the plurality of types of consumable.
Clause 61. The method of clause 59 or clause 60, wherein the plurality of types of consumable are externally identical but differ in the electrical characteristic, and in volume of the aerosol-generating material that the plurality of types of consumable are structured to receive.
Clause 62. The method of any of clauses 59 to 61, wherein the different levels of power are for the different amounts of aerosol that are measured in terms of aerosol collected mass (ACM) or total particulate matter (TPM).
Clause 63. The method of any of clauses 59 to 62, wherein the aerosol generator is a resistive heating element, and the different levels of power are for different temperatures to which the resistive heating element is driven to generate the different amounts of aerosol.
Clause 64. The method of any of clauses 59 to 63, wherein the method further comprises setting the aerosol provision device to the one of the power modes before power is provided to the aerosol generator.
Clause 65. The method of any of clauses 59 to 64, wherein the power provided to the aerosol generator is controlled responsive to user input that indicates a draw on the consumable.
Clause 66. The method of any of clauses 59 to 65, wherein the coupler includes electrical connectors configured to electrically couple the aerosol provision device with the consumable, and wherein the electrical characteristic of the consumable is measured at the electrical connectors.
Clause 67. The method of clause 66, wherein measuring the electrical characteristic of the consumable includes at least: measuring at least one of a voltage across or a current through the consumable at the electrical connectors; and determining the value of the electrical characteristic of the consumable based on the at least one of the voltage or the current.
Clause 68. The method of clause 67, wherein the voltage across the consumable is measured from a positive one of the electrical connectors and referenced to ground.
Clause 69. The method of clause 67 or clause 68. wherein the current through the consumable is measured using a low-side current-sensing circuit coupled to a negative one of the electrical connectors.
Clause 70. The method of any of clauses 67 to 69, wherein the electrical characteristic is electrical resistance, and the value of the electrical characteristic is a value of the electrical resistance of the consumable, and wherein the voltage across and the current through the consumable are measured, and the value of the electrical resistance of the consumable is determined based on the voltage and the current.
Clause 71. The method of any of clauses 59 to 70, wherein the electrical characteristic is electrical resistance, the target values are target electrical resistance values, the value of the electrical characteristic is a value of the electrical resistance of the consumable, and the one of the target values is one of the target electrical resistance values.
Clause 72. The method of clause 71, wherein the method further comprises confirming the value of the electrical resistance of the consumable is above a threshold resistance value that indicates a short-circuit condition of the aerosol provision device, before the one of the one of the target electrical resistance values is identified.
Clause 73. The method of clause 71 or clause 72, wherein the aerosol generator is a resistive heating element, and the electrical resistance of the consumable is the electrical resistance of the resistive heating element in a lumped-element model of the consumable.
Clause 74. The method of any of clauses 71 to 73, wherein the aerosol generator is a resistive heating element, the consumable further includes a series resistor, and the electrical resistance of the consumable is a sum of the electrical resistance of the resistive heating element and the series resistor in a lumped-element model of the consumable.
Clause 75. The method of any of clauses 59 to 74, wherein the electrical characteristic is current, the target values are target current values, the value of the electrical characteristic is a value of the current through the consumable, and the one of the target values is one of the target current values.
Clause 76. The method of clause 75, wherein the method further comprises confirming the value of the current through the consumable is below a threshold current value that indicates a short-circuit condition of the aerosol provision device, before the one of the one of the target electrical resistance values is identified.
Clause 77. The method of clause 75 or clause 76, wherein the aerosol generator is a resistive heating element, and the current through the consumable is the current through the resistive heating element in a lumped-element model of the consumable.
Clause 78. The method of any of clauses 59 to 77, wherein the electrical characteristic is current, the target values are target current waveforms, the value of the electrical characteristic is a waveform of the current through the consumable, and the one of the target values is one of the target current waveforms.
Clause 79. The method of clause 78, wherein the aerosol generator is a resistive heating element; the consumable further includes a resistor-capacitor (RC) circuit that includes the resistive heating element, and the waveform of the current through the consumable is a waveform of the current through the RC circuit in a lumped-element model of the consumable.
Clause 80. The method of clause 78 or clause 79, wherein the aerosol generator is a resistive heating element, the consumable further includes a resistor-inductor-capacitor (RLC) circuit that includes the resistive heating element, and the waveform of the current through the consumable is a waveform of the current through the RLC circuit in a. lumped-element model of the consumable.
Clause 81. The method of any of clauses 59 to 80, wherein the consumable is of a first of the plurality of types of consumable having a first target value of the electrical characteristic associated with a first power mode of a first level of power; a second consumable of a second of the plurality of types of consumable having a second target value of the electrical characteristic associated with a second power mode of a second level of power.
Clause 82. The method of clause 81, wherein the first of the plurality of types of consumable is structured to receive a first volume of the aerosol-generating material, and the second of the plurality of types of consumable is structured to receive a second volume of the aerosol-generating material.
Clause 83. The method of clause 82, wherein the first volume of the aerosol-generating material is greater than the second volume, and the first value of the power setting is greater than the second value.
Clause 84. The method of any of clauses 81 to 83, wherein the method further comprises: measuring the electrical characteristic of the second consumable engaged with the coupler and thereby the aerosol provision device, the electrical characteristic of the second consumable measured to obtain a second value of the electrical characteristic; identifying the second target value that most closely matches the second value of the electrical characteristic of the second consumable, the second target value associated with the second power mode of the second level of power; and controlling the power provided to the aerosol generator to the second level of power to energize the aerosol-generating material of the second consumable to generate aerosol for delivery to the user.
Clause 85. The method of any of clauses 59 to 84, wherein the aerosol provision device further comprises a power source configured to provide a source of power to the aerosol provision device, and wherein controlling the power provided to the aerosol generator switchable connecting and disconnecting the power source and the aerosol generator.
Clause 86. The method of any of clauses 59 to 85, wherein the controlling the power provided to the aerosol generator includes at least: measuring a voltage across and a current through the aerosol generator; calculating the power provided to the aerosol generator based on the voltage and the current; and adjusting the power provided to the aerosol generator at each instance in which the power as calculated deviates from the respective one of the levels by more than a threshold power.
Clause 87. The method of clause 86, wherein calculating the power provided to the aerosol generator includes at least: determining a moving window of measurements of instantaneous actual power provided to the aerosol generator, each measurement of the window of measurements determined based on the voltage and the current; and calculating the power provided to the aerosol generator as a simple moving average power based on the moving window of measurements instantaneous actual power.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

What is claimed is:

1. An aerosol provision device comprising:
a coupler structured to engage a consumable with an aerosol generator and aerosol-generating material, the consumable one of a plurality of types of consumable having target values of an electrical characteristic that are associated with respective power modes of different levels of power for different amounts of aerosol generated from the aerosol-generating material; and
circuitry that includes processing circuitry configured to control power provided to the aerosol generator to energize the aerosol-generating material of the consumable to generate aerosol for delivery to a user, including the processing circuitry configured to at least:
measure the electrical characteristic of the consumable to obtain a value of the electrical characteristic;
identify one of the target values that most closely matches the value of the electrical characteristic of the consumable, the one of the target values associated with one of the power modes of a respective one of the different levels of power for a respective one of the different amounts of aerosol; and
control the power provided to the aerosol generator to the respective one of the different levels of power for the respective one of the different amounts of aerosol.

2. The aerosol provision device of claim 1, wherein the coupler includes electrical connectors configured to electrically couple the circuitry and thereby the aerosol provision device with the consumable, and
wherein the processing circuitry is configured to measure the electrical characteristic of the consumable at the electrical connectors, including the processing circuitry configured to at least:
measure at least one of a voltage across or a current through the consumable at the electrical connectors; and
determine the value of the electrical characteristic of the consumable based on the at least one of the voltage or the current.

3. The aerosol provision device of claim 1, wherein the electrical characteristic is electrical resistance, the target values are target electrical resistance values, the value of the electrical characteristic is a value of the electrical resistance of the consumable, and the one of the target values is one of the target electrical resistance values.

4. The aerosol provision device of claim 3, wherein the processing circuitry is further configured to confirm the value of the electrical resistance of the consumable is above a threshold resistance value that indicates a short-circuit condition of the aerosol provision device, before the one of the one of the target electrical resistance values is identified.

5. The aerosol provision device of claim 1, wherein the electrical characteristic is current, the target values are target current values, the value of the electrical characteristic is a value of the current through the consumable, and the one of the target values is one of the target current values.

6. The aerosol provision device of claim 1, wherein the electrical characteristic is current, the target values are target current waveforms, the value of the electrical characteristic is a waveform of the current through the consumable, and the one of the target values is one of the target current waveforms.

7. The aerosol provision device of claim 1, wherein the consumable is of a first of the plurality of types of consumable having a first target value of the electrical characteristic associated with a first power mode of a first level of power, a second consumable of a second of the plurality of types of consumable having a second target value of the electrical characteristic associated with a second power mode of a second level of power.

8. The aerosol provision device of claim 7, wherein the first of the plurality of types of consumable is structured to receive a first volume of the aerosol-generating material, and the second of the plurality of types of consumable is structured to receive a second volume of the aerosol-generating material.

9. The aerosol provision device of claim 7, wherein the processing circuitry is further configured to at least:
measure the electrical characteristic of the second consumable engaged with the coupler and thereby the aerosol provision device, the electrical characteristic of the second consumable measured to obtain a second value of the electrical characteristic;
identify the second target value that most closely matches the second value of the electrical characteristic of the second consumable, the second target value associated with the second power mode of the second level of power; and
control the power provided to the aerosol generator to the second level of power to energize the aerosol-generating material of the second consumable to generate aerosol for delivery to the user.

10. The aerosol provision device of claim 1, wherein the processing circuitry configured to control the power provided to the aerosol generator includes the processing circuitry configured to at least:
measure a voltage across and a current through the aerosol generator;
calculate the power provided to the aerosol generator based on the voltage and the current; and
adjust the power provided to the aerosol generator at each instance in which the power as calculated deviates from the respective one of the levels by more than a threshold power.

11. A method of operating an aerosol provision device that includes a coupler structured to engage a consumable with an aerosol generator and aerosol-generating material, the consumable one of a plurality of types of consumable having target values of an electrical characteristic that are associated with respective power modes of different levels of power for different amounts of aerosol generated from the aerosol-generating material, the method comprising:
controlling power provided to the aerosol generator to energize the aerosol-generating material of the consumable to generate aerosol for delivery to a user, including at least:
measuring the electrical characteristic of the consumable to obtain a value of the electrical characteristic;
identifying one of the target values that most closely matches the value of the electrical characteristic of the consumable, the one of the target values associated with one of the power modes of a respective one of the different levels of power for a respective one of the different amounts of aerosol; and
controlling the power provided to the aerosol generator to the respective one of the different levels of power for the respective one of the different amounts of aerosol.

12. The method of claim 11, wherein the coupler includes electrical connectors configured to electrically couple the aerosol provision device with the consumable, and
wherein the electrical characteristic of the consumable is measured at the electrical connectors, including at least:
measuring at least one of a voltage across or a current through the consumable at the electrical connectors; and
determining the value of the electrical characteristic of the consumable based on the at least one of the voltage or the current.

13. The method of claim 11, wherein the electrical characteristic is electrical resistance, the target values are target electrical resistance values, the value of the electrical characteristic is a value of the electrical resistance of the consumable, and the one of the target values is one of the target electrical resistance values.

14. The method of claim 13, wherein the method further comprises confirming the value of the electrical resistance of the consumable is above a threshold resistance value that indicates a short-circuit condition of the aerosol provision device, before the one of the one of the target electrical resistance values is identified.

15. The method of claim 11, wherein the electrical characteristic is current, the target values are target current values, the value of the electrical characteristic is a value of the current through the consumable, and the one of the target values is one of the target current values.

16. The method of claim 11, wherein the electrical characteristic is current, the target values are target current waveforms, the value of the electrical characteristic is a waveform of the current through the consumable, and the one of the target values is one of the target current waveforms.

17. The method of claim 11, wherein the consumable is of a first of the plurality of types of consumable having a first target value of the electrical characteristic associated with a first power mode of a first level of power, a second consumable of a second of the plurality of types of consumable having a second target value of the electrical characteristic associated with a second power mode of a second level of power.

18. The method of claim 17, wherein the first of the plurality of types of consumable is structured to receive a first volume of the aerosol-generating material, and the second of the plurality of types of consumable is structured to receive a second volume of the aerosol-generating material.

19. The method of claim 17, wherein the method further comprises:
measuring the electrical characteristic of the second consumable engaged with the coupler and thereby the aerosol provision device, the electrical characteristic of the second consumable measured to obtain a second value of the electrical characteristic;
identifying the second target value that most closely matches the second value of the electrical characteristic of the second consumable, the second target value associated with the second power mode of the second level of power; and
controlling the power provided to the aerosol generator to the second level of power to energize the aerosol-generating material of the second consumable to generate aerosol for delivery to the user.

20. The method of claim 11, wherein the controlling the power provided to the aerosol generator includes at least:
measuring a voltage across and a current through the aerosol generator;
calculating the power provided to the aerosol generator based on the voltage and the current; and
adjusting the power provided to the aerosol generator at each instance in which the power as calculated deviates from the respective one of the levels by more than a threshold power.
